(19) United States
(12) Patent Application Publication
(10) Pub. No.: US 2012/0022461 A1
(43) Pub. Date: Jan. 26, 2012

(54) TILTABLE SAFETY NEEDLE

(75) Inventors: Jakob Bøgh Schubert, Graested (DK); Kristian Glejbøl, Glostrup (DK); Martin Von Bülow, Helsingor (DK)

(73) Assignee: NOVO NORDISK A/S

(21) Appl. No.: 13/139,420

(22) PCT Filed: Dec. 1, 2009
(86) PCT No.: PCT/EP2009/066130

§ 371 Date: Sep. 29, 2011

Related U.S. Application Data
(60) Provisional application No. 61/138,288, filed on Dec. 17, 2008.

(30) Foreign Application Priority Data
Dec. 15, 2008 (EP) 08171631.8

(51) Int. Cl. A61M 5/32

(52) U.S. Cl. 604/192

(57) ABSTRACT

A shielded needle assembly for a drug delivery device in which a needle cannula (20) tilts once the needle assembly is removed from the drug delivery device whereby a non-patient end (22) of a needle cannula (20) abuts an inside surface of the needle hub (1) and a patient end (21) of the same needle cannula (20) blocks the axial movement of the shield (50).

### THE TECHNICAL FIELD OF THE INVENTION

The invention relates to a needle assembly having a shielded needle cannula and especially a pen needle assembly where both ends of the needle cannula are secured after use.

### DESCRIPTION OF RELATED ART

People suffering from diabetes have to inject themselves with insulin at a daily basis. For this purpose a great number of different pen systems have been developed over the last 30 years. Common for pen injectors is that they contain a container or cartridge containing the liquid drug to be injected. In order to transfer the liquid drug from the injection pen and into the body of the patient a pen needle assembly is used. Such pen-needle assemblies have a needle cannula with a non-injection end which penetrates into the cartridge when the pen needle assembly is attached to the injection pen and a patient end that enters into the body of the patient during injection to create a liquid communication between the inner of the cartridge and the patient. A pen needle assembly further comprises a hub carrying the cannula and which hub is usually provided with means for attaching the pen needle assembly to the injection pen.

Such pen needle assemblies are typically disposable and are discarded after one single use. The problem presented by the disposal of a pen needle assembly, and indeed, by any handling of the pen needle assembly, is the potential risk of being injured by any of the sharp ends of the needle cannula. This is particular dangerous when following after the penetration of a patients skin since the needle cannula then may be contaminated and therefore capable of spreading diseases such as hepatitis and HIV.

A great number of pen needle assemblies have been developed where the patient end of the needle cannula is concealed by a spring loaded and telescopically movable shield during and after the injection. Such safety needles are e.g. known from WO 03/066141, EP 1.289.587 and in EP 1.448.256.

A common feature with all these known pen needle assemblies is however that the non-injection end of the needle cannula is not concealed but has its sharp point located only a few millimetres below the edge of the skirt surrounding the non-injection end. The distance between the sharp point of the non-injection end of the needle cannula and the edge of the hub must be very little in order for the non-injection end of the needle to penetrate properly into the cartridge when the hub is attached to an injection pen. This relatively short distance can result in accidental needle stick injuries. This is especially possible if the person handling the used needle assembly has small fingers that can be squeezed into the area within the skirt of the hub e.g. when returning the needle assembly to the outer container after use.

In order to protect the user from such accidentally injuries with the back-needle, a number of pen needles with back-needle protection has been suggested e.g. in WO 2008/107199 and in U.S. Pat. No. 7,384,414.

Further, a number of safety pen needles concealing both the injection-end and the non-injection end of the needle cannula at the same time have been disclosed in US 2008/0177235, US 2008/0177237 and US 2008/0177238. These constructions are however very complicated and requires several springs in order to function.

### DESCRIPTION OF THE INVENTION

It is an object of the present invention to provide a needle assembly having a shielded needle cannula and wherein both the patient end and the non-patient end of the needle cannula can be secured and which needle assembly is both cheap to manufacture and reliable to use.

### Claim 1

In its first position the patient end of the needle cannula points straight forward along the centre axis of the needle assembly. In this position, the needle assembly is ready for use and the shield can slide parallel to the needle cannula.

Once the needle cannula has been moved to its second position which usually follows use of the needle assembly, the needle cannula is moved to a second position wherein the needle cannula is not parallel aligned with the centre axis (x) and in which position the shield prevents exposure of the tip of the needle cannula (20) while the patient end of the needle cannula blocks the axial movement of the shield.

Since the patient end and the non-patient end of the needle cannula follow each other, the non-patient end is also deflected. In its deflected position the sharp end is pushed against the skirt of the hub thereby rendering it impossible—or at least very difficult—to be injured by the sharp end of the non-patient end. The hub could alternatively be provided with a radial extension into which the non-patient end collapses.

### Claim 2-6

The needle cannula is preferably mounted, e.g. by gluing, to an attachment part which is connected to a bridging part suspended over the hub. The bridging part is firmly attached to the hub at one end while the other end is free-floating. The bridging part is preferably moulded as an integral part of the hub but could also be a separate part attached to the hub.

This free floating end of the bridging portion is preferably supported by a part of the hub which can be moved out of engagement with the free floating end of the bridging part in which situation the bridge would collapse resulting in a deflection of the needle cannula.

The part of the hub which supports the free floating end of the bridging portion is preferably a radially movable part of the hub. It can be embodied as a tooth or a cut-out in the skirt of the hub and only being proximally connected to the hub, or it can be embodied as an insert in the hub. The function is that it supports the free floating end of the bridging portion when the needle assembly is not attached to the injection device but is automatically shifted out of engagement with the free floating end once the needle assembly is attached to any injection device. However, the release part could also be a part that requires the user to actuate it.

In order to enhance the collapse of the bridging part it is preferably provided with a film hinge or a similar mechanism that weakens the structure.

### Claim 7-8

To further enhance the collapse a spring is provided which urges the bridging part in the direction of the collapse which is preferably the proximal direction.

In order to have the telescopically movable shield move into its covering position after an injection, a spring is provided which urges the shield in the distal direction. This spring is preferably provided between the shield and the bridging portion of the hub such that the same spring urges the shield in the distal direction and applies a pressure on the bridging portion enhancing the collapse once the free floating end is set free.

### Claim 9-10

Once the needle assembly is removed from the injection device the free floating end of the bridging structure of the hub is no longer supported which makes it collapse. Since the part of the bridging structure opposite the free floating part is secured to the hub, the bridge with its attachment part will tilt. The needle cannula which is secured to the attachment part will also tilt whereby the patient end of the needle cannula will be deflected away form the centre axis and the non-patient end will be deflected away in the opposite direction.

The sharp end of the non-patient end will abut the inside wall of the skirt of the hub while the sharp part of the patient end will be positioned inside the shield thereby preventing axial movement of the shield.

### DEFINITIONS

An “injection pen” is typically an injection apparatus having an oblong or elongated shape somewhat like a pen for writing. Although such pens usually have a tubular cross-section, they could easily have a different cross-section such as triangular, rectangular or square or any variation around these geometries.

As used herein, the term “drug” is meant to encompass any drug-containing flowable medicine capable of being passed through a delivery means such as a hollow needle in a controlled manner, such as a liquid, solution, gel or fine suspension. Representative drugs includes pharmaceuticals such as peptides, proteins (e.g. insulin, insulin analogues and C-peptide), and hormones, biologically derived or active agents, hormonal and gene based agents, nutritional formulas and other substances in both solid (dispensed) or liquid form.

Correspondingly, the term “subcutaneous” injection is meant to encompass any method of transcutaneous delivery to a subject.

The term “Needle Cannula” is used to describe the actual conduit performing the penetration of the skin during injection. A needle cannula is usually made from a metallic material and connected to a hub to form a complete injection needle also often referred to as a “needle assembly”. A needle cannula could however also be made from a polymeric material or a glass material. The needle cannula is mounted in a “hub”, which also carries the connecting means for connecting the injection needle to an injection apparatus and is usually moulded from a suitable thermoplastic material. The “connection means” could as examples be a luer coupling, a bayonet coupling, a threaded connection or any combination thereof e.g. a combination as described in EP 1,536,854.

“Cartridge” is the term used to describe the container containing the drug. Cartridges are usually made from glass but could also moulded from any suitable polymer. A cartridge or ampoule is preferably sealed at one end by a pierceable membrane which can be pierced e.g. by the non-patient end of a needle cannula. The opposite end is typically closed by a plunger or piston made from rubber or a suitable polymer. The plunger or piston can be slidable moved inside the cartridge. The space between the pierceable membrane and the movable plunger holds the drug which is pressed out as the plunger decreased the volume of the space holding the drug. However, any kind of container—rigid or flexible—can be used to contain the drug.

All references, including publications, patent applications, and patents, cited herein are incorporated by reference in their entirety and to the same extent as if each reference were individually and specifically indicated to be incorporated by reference and were set forth in its entirety herein.

All headings and sub-headings are used herein for convenience only and should not be constructed as limiting the invention in any way.

The use of any and all examples, or exemplary language (e.g. such as) provided herein, is intended merely to better illuminate the invention and does not pose a limitation on the scope of the invention unless otherwise claimed. No language in the specification should be construed as indicating any non-claimed element as essential to the practice of the invention. The citation and incorporation of patent documents herein is done for convenience only and does not reflect any view of the validity, patentability, and/or enforceability of such patent documents.

This invention includes all modifications and equivalents of the subject matter recited in the claims appended hereto as permitted by applicable law.

### BRIEF DESCRIPTION OF THE DRAWINGS

The invention will be explained more fully below in connection with a preferred embodiment and with reference to the drawings in which:

FIG. 1 Show an exploded view of the tiltable needle assembly.

FIG. 2 Show a cross sectional view of the tiltable needle assembly prior to mounting.

FIG. 3 show a cross sectional view of the tiltable needle cannula in its locked position after removal from the injection pen.

FIG. 4 Show a cross sectional view of the tiltable needle assembly mounted an injection pen.

The figures are schematic and simplified for clarity, and they just show details, which are essential to the understanding of the invention, while other details are left out. Throughout, the same reference numerals are used for identical or corresponding parts.

### DETAILED DESCRIPTION OF EMBODIMENT

When in the following terms as “upper” and “lower”, “right” and “left”, “horizontal” and “vertical”, “clockwise” and “counter clockwise” or similar relative expressions are used, these only refer to the appended figures and not to an actual situation of use. The shown figures are schematic representations for which reason the configuration of the different structures as well as there relative dimensions are intended to serve illustrative purposes only.

In that context it may be convenient to define that the term “distal end” in the appended figures is meant to refer to the end of the needle cannula penetrating the patient whereas the term “proximal end” is meant to refer to the opposite end pointing away from the patient in a situation of use.

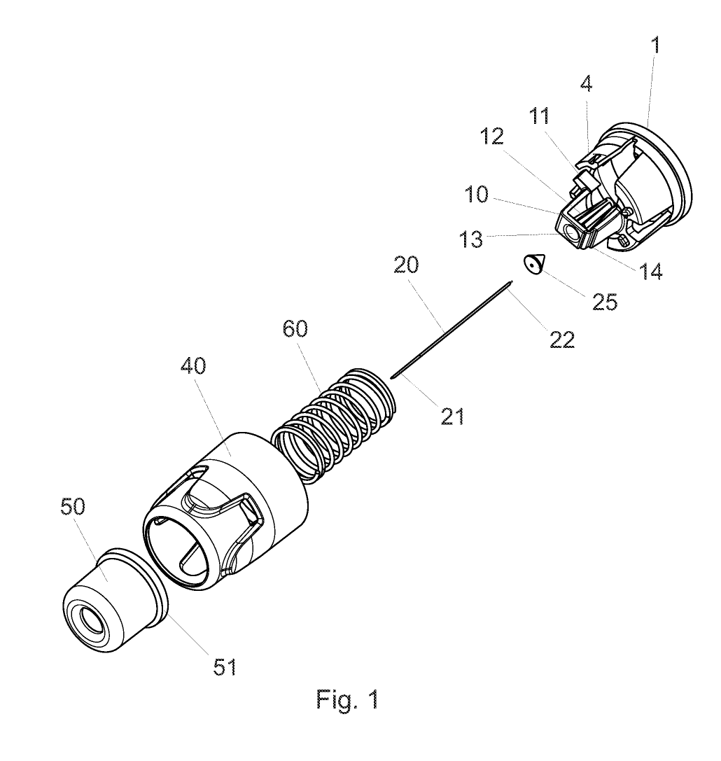

The needle assembly disclosed in FIG. 1 comprises a needle cannula 20 which is glued to a needle hub 1 by a drop of glue 25. The needle cannula 20 comprises a distal part 21 for penetrating into a patient (the patient end) and a proximal part 22 for penetrating into a cartridge 31 (the non-patient end) contained in the injection device 30 to which the needle assembly is attached.

Surrounding the hub 1 is a tubular housing 40 which is preferably glued or welded to the outside surface of the hub 1. A safety shield 50 is axially movable inside the housing 40 and is urged in the distal direction by a spring 60 provided between the hub 1 and the shield 50.

The shield 50 is at its proximal end provided with a rim 51 which engages an inwardly pointing edge 41 on the housing 40 such that the shield 50 is contained within the housing 40.

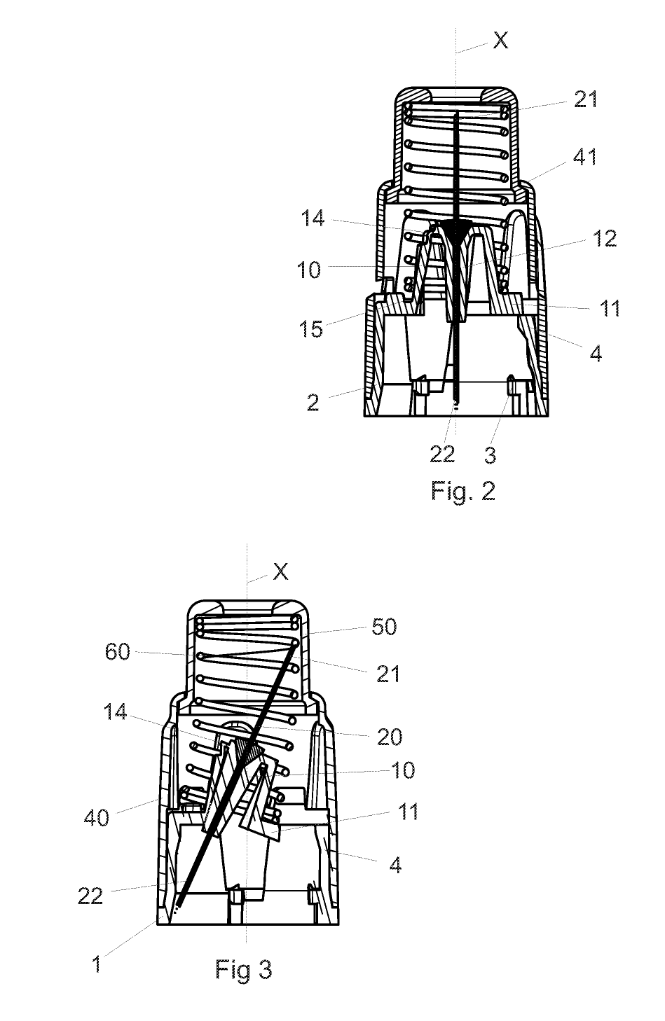

As disclosed in FIGS. 2 and 3 the hub 1 comprises a number of inwardly pointing protrusions 3 located on the inside surface of a skirt 2 surrounding the proximal part 22 of the needle cannula 20, such that the needle assembly can be attached to the injection device 30 via a bayonet interface as described in EP 1,536,854. Such interface only requires the user to perform a relatively little rotation of the needle assembly relatively to the injection device 30 in order to attach the needle assembly properly.

The hub 1 further comprises a bridge 10 which at one side is firmly attached to the hub 1 e.g. by being moulded as an integral part of the hub 1. At the other end, approximately 180 degrees opposite from the first side, the bridge 10 terminates in a free-floating leg 11.

The bridge 10 carries an attachment part 12 with a through-going opening 13, in which opening 13 the needle cannula 20 is glued 25 to the bridge 10. The bridge 10 further comprises a film-hinge 14 which is preferably moulded as an integral part of the bridge 10. Further the hub 1 and the bridge 10 is provided with a track 15 which supports the spring 60.

The skirt 2 of the hub 1 comprises a release part 4 which is radially movable relatively to the remaining part of the skirt 2. This release part 4 supports the free-floating leg 11 of the bridge 10 at its distal end as disclosed in FIG. 2.

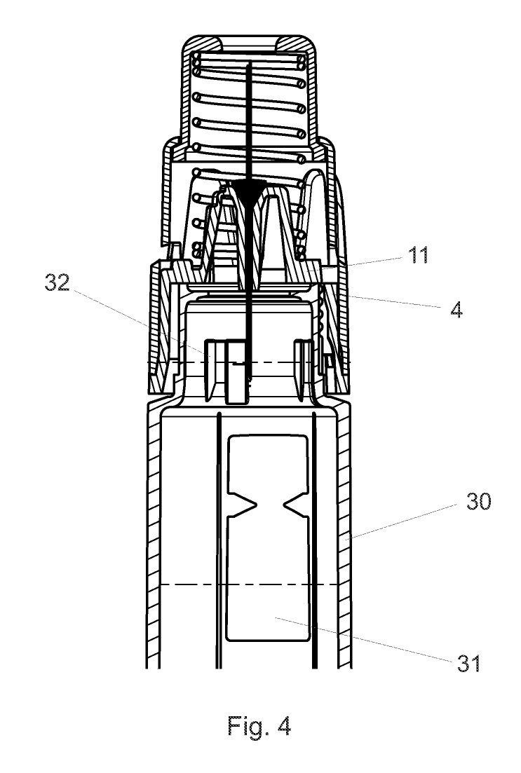

FIG. 4 discloses the needle assembly attached to the injection device 30 utilizing the bayonet interface. The injection device 30 supports the cartridge 31 holding the drug to be injected and is at its distal end provided with a needle mount 32. This needle mount 32 has a number of axial tracks for the bayonet coupling and can either be a part of the injection device 30 or a separate part attached to the cartridge 31.

Once the needle assembly is attached to the injection device 30, the release part 4 is moved radially outwardly by the needle mount 32. In this mode the injection pen 30 is ready to perform the injection and the free-floating leg 11 of the bridge 10 is supported by the needle mount 32.

When a user performs an injection he or she simply pushes the injection device 30 with the needle assembly attached against the surface of the skin whereby the skin moves the shield 50 in the proximal direction against the pressure of the spring 60. When the injection is over the user removes the injection device 30 together with the needle assembly whereby the spring 60 moves the shield 50 in the distal direction. The patient end 21 of the needle cannula 20 will at all time be out of the sight of the user.

When the user decides to remove the needle assembly from the injection device 30 he or she uncouples the needle assembly as disclosed in FIG. 3. Since the free-floating leg 11 is now unsupported the spring 60 which is located on the bridge 10 pushes the bridge 10 in the proximal direction. The film-hinge 14 now bends to the pressure of the spring 60 and the entire bridge 10 including the attachment part 12 and the needle cannula 20 tilts.

In this tilted position disclosed in FIG. 3, the non-patient end of the needle cannula lies against the inside surface of the skirt 2 and the patient end 21 of the needle cannula 20 blocks the shield 50 such that the shield 50 can not be moved axially.

Some preferred embodiments have been shown in the foregoing, but it should be stressed that the invention is not limited to these, but may be embodied in other ways within the subject matter defined in the following claims, e.g. could a needle assembly as herein described be delivered to the user in a rigid and sterile container which further could be shaped as a tool for assisting the user in mounting the needle assembly on to the injection device.

1. A needle assembly for a drug delivery device comprising
A needle hub (1) attachable to the drug delivery device (30),
A needle cannula (20) secured to the hub (1) and having a patient end (21) and a non-patient end (22),
A telescopically movable shield (50) adapted to at least partially cover the patient-end (21) of the needle cannula (20),
Characterized in that, the needle cannula (20) has a first position in which the needle cannula (20) is substantial parallel aligned with a centre axis (X) of the needle assembly, and a second position in which the needle cannula (20) deflects from the first position and in which second position the patient end (21) of the needle cannula (20) engages the shield (50) whereby exposure of the patient end (21) of the needle cannula (20) is prevented.

2. A needle assembly for a drug delivery device according to claim 1, in which the needle hub (1) comprises a bridge part (10) holding the needle cannula (20).

3. A needle assembly for a drug delivery device according to claim 2, in which the bridge part (10) has a free-floating leg (11).

4. A needle assembly for a drug delivery device according to claim 3, in which the free-floating leg (11) is supported by a release part (4).

5. A needle assembly for a drug delivery device according to claim 4, in which the release part (4) is a radially movable part of the hub (1).

6. A needle assembly for a drug delivery device according to claim 5, in which the bridge part (10) is provided with a film hinge (14).

7. A needle assembly for a drug delivery device according to claim 2, in which a spring (60) is provided which applies a pressure on the bridge part (10) in a proximal direction.

8. A needle assembly for a drug delivery device according to claim 7, in which the spring (60) is provided between the bridge part (10) and the shield (50).

9. A needle assembly for a drug delivery device according to claim 4, in which the release part (4) is moved in a radial direction by a needle mount (32) of the injection device (30) to which the needle assembly is attached.

10. A needle assembly for a drug delivery device according to claim 9, in which a non-patient end (22) of the needle cannula (20) abuts an inside surface of the needle hub (1) and a patent-end (21) of the needle cannula (20) blocks the axial movement of the shield (50) once the needle assembly has been removed from the injection device (30).
